(19) United States
(12) Patent Application Publication
(10) Pub. No.: US 2015/0072404 A1
(43) Pub. Date: Mar. 12, 2015

(54) EXAMINING TEST PAPER

(71) Applicant: Chun-Tung (Jack) CHEN, Taipei City (TW)

(72) Inventor: Chun-Tung (Jack) CHEN, Taipei City (TW)

(21) Appl. No.: 14/314,868

(22) Filed: Jun. 25, 2014

(30) Foreign Application Priority Data
Sep. 12, 2013 (TW) 102217160

(51) Int. Cl. C12Q 1/00

(52) U.S. Cl. CPC C12Q 1/006
USPC 435/287.8

(57) ABSTRACT

The invention is related to an examining test paper which comprises an upper substrate, a lower substrate, an upper insulation sheet, a lower insulation sheet and a separator. Each of the upper and lower substrates comprises surface, a notch, a connection area, a reaction area, and an electrode layer. The notch is recessed on a proximal end. The connection area is adjacent to the notch. The reaction area and the electrode layer are defined on the surface. The upper and lower insulation sheets are respectively connected to the upper and lower substrates, and each comprises an upper surface, a lower surface, and a permeation portion. The separator is connected to the upper and lower insulation sheets and comprises two permeation portions corresponding to the permeation portions of the upper and lower insulation sheets, respectively.

The current application claims a foreign priority to the patent application of Taiwan No. 102217160 filed on Sep. 12, 2013.

### BACKGROUND OF THE INVENTION

1. Field of the Invention

The present invention relates to a test paper, especially to a test paper used for sampling blood and examining blood glucose concentration.

2. Description of the Prior Art

A blood glucose meter sold on markets is used for examining the blood glucose concentration in human blood. An examining test of the blood glucose concentration requires a drop of blood of the individual to be examined on a specific site of a test paper, as an input to a blood glucose meter which reads and indicate the blood glucose concentration of the individual. The test paper comprises an electrode layer and a reaction area. The reaction area is defined on a surface of the test paper. A front of the electrode layer is located in the reaction area, whereas an end of the electrode layer is to be connected to the blood glucose meter. The reaction area of the test paper comprises a superficial enzyme layer. The enzyme layer comprises at least one enzyme that reacts with the blood sample to generate signals which the blood glucose meter detects through the electrode layer.

A conventional test paper is of a single-layer structure and capable of performing merely one single test. A duplication of tests for increasing accuracy or an extra test of different categories for broader knowledge of the individual demands an additional test paper, as well as an additional drop of blood sample, and thus is significantly inconvenient to perform.

To overcome the shortcomings, the present invention provides a test paper comprising a double-layer structure to enhance examining accuracy or to simultaneously perform two different kinds of biochemical examination.

### SUMMARY OF THE INVENTION

The examining test paper in accordance with the present invention comprises an upper substrate, a lower substrate, an upper insulation sheet, a lower insulation sheet and a separator. Each of the upper and lower substrates comprises surface, a notch, a connection area, a reaction area, and an electrode layer. The notch is recessed on a proximal end. The connection area is adjacent to the notch. The reaction area and the electrode layer are defined on the surface. The upper and lower insulation sheets are respectively connected to the upper and lower substrates, and each comprises an upper surface, a lower surface, and a permeation portion. The separator is connected to the upper and lower insulation sheets and comprises two permeation portions corresponding to the permeation portions of the upper and lower insulation sheets, respectively.

The present invention at least provides the following improvements. Setting the electrode layer and the reaction area on the upper and lower substrate, and further setting an enzyme layer on the reaction areas help to examine blood glucose. Structures of the notches recessed on the upper and lower substrates are helpful for combination of the upper and lower substrate and corresponding to a circumscribed convert connector. Connecting the upper and lower insulation sheets respectively to the surfaces of the upper and lower substrates, helps to avoid examining errors caused by contact of the electrode layers of the upper and lower substrates. Using a separator connected to the upper insulation sheet and the lower insulation sheet allows the examining test paper to perform multiple examinations simultaneously. Two examining results can be obtained with one single drop of blood on the examining test paper once, and users can confirm the accuracy of blood glucose examination from the two examining results.

Furthermore, a GOD/GDH enzyme layer is applied to the reaction area of the upper substrate and an impedance membrane is formed on the reaction area of the lower substrate to enhance the accuracy of blood glucose examination.

Furthermore, the GOD/GDH enzyme layer and another enzyme layer are applied to the reaction areas of the upper substrate and the lower substrate, respectively, to examine different blood compositions.

### BRIEF DESCRIPTION OF THE DRAWINGS

FIG. 1 is a three-dimensional view of the decomposed components in accordance with the present invention.

FIG. 2 is an allocated figure in accordance with the present invention.

FIG. 3 is a usage stage figure in accordance with the present invention.

FIG. 4 is a decomposition chart of the second embodiment in accordance with the present invention.

FIG. 5 is a decomposition chart of the third embodiment in accordance with the present invention.

### DETAILED DESCRIPTION OF THE PREFERRED EMBODIMENTS

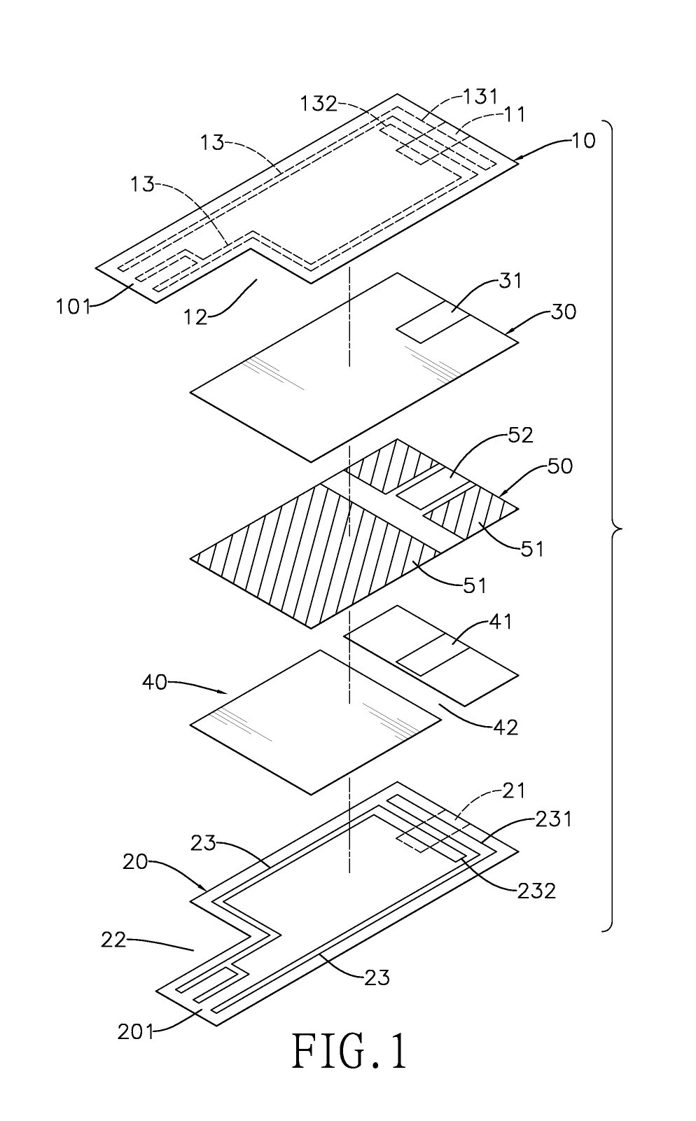

With reference to FIG. 1, in the first embodiment, an examining test paper in accordance with the present invention comprises an upper substrate 10, a lower substrate 20, an upper insulation sheet 30, a lower insulation sheet 40 and a separator 50.

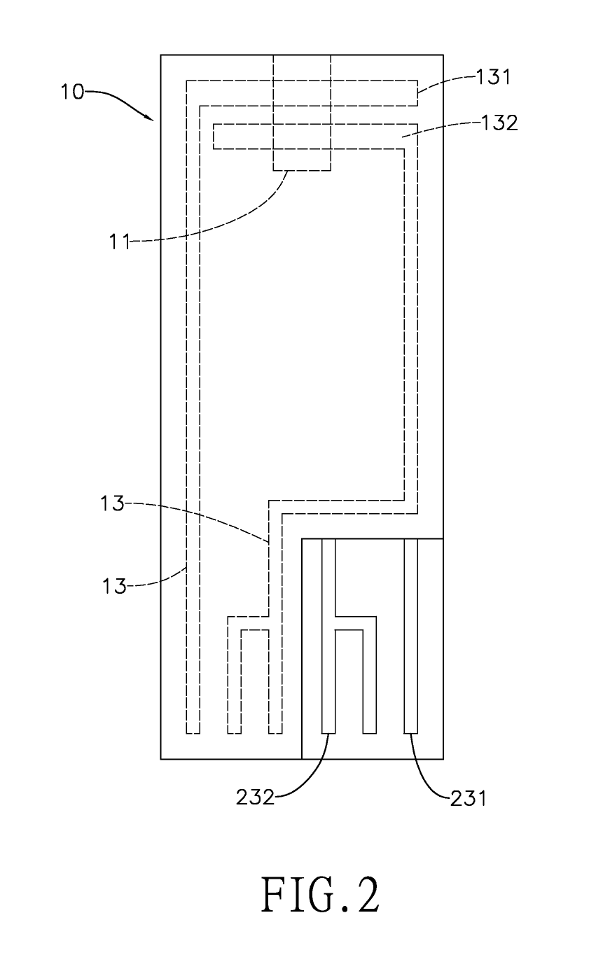

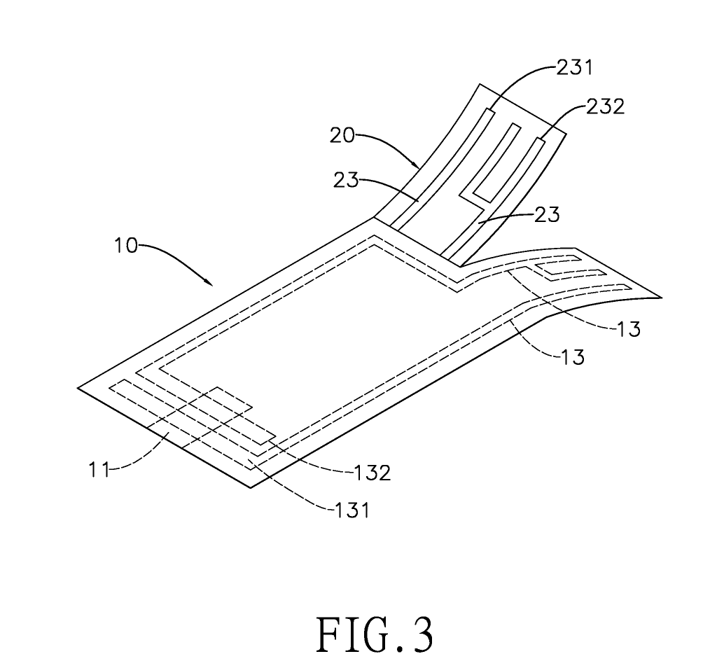

With reference to FIGS. 1 to 3, the upper substrate 10 is a thin film and comprises a surface, a proximal end, a distal end, a notch 12, a connection area 101, a reaction area 11, and an electrode layer 13. The distal end is opposite to the proximal end. The notch 12 is recessed on the proximal end of the upper substrate 10. The connection area 101 is formed on the upper substrate 10 adjacent to the notch 12. The reaction area 11 is defined on the surface near the distal end of the upper substrate 10 and comprises a hydrophilic layer. The electrode layer 13 is defined on the surface of the upper substrate 10, and comprises a transmitting electrode 131 and a reference electrode 132. The transmitting electrode 131 which is a transmitting end and arranged in accordance with a shape of the upper substrate 10 comprises a distal portion overlapped with the reaction area 11 of the upper substrate 10, and a proximal portion located at the connection area 101 of the upper substrate 10. The reference electrode 132 which is a grounding terminal and arranged separately from the transmitting electrode 131 in accordance with a shape of the upper substrate 10 comprises a distal portion overlapped with the reaction area 11 of the upper substrate 10, and a proximal portion located at the connection area 101 of the upper substrate 10. The lower substrate 20 is a thin film and comprises a surface, a proximal end, a distal end, a notch 22, a connection area 201, a reaction area 21, and an electrode layer 23. The distal end is opposite to the proximal end. The notch 22 is recessed on the proximal end of the lower substrate 20. The connection area 201 is formed on the lower substrate 20 adjacent to the notch 22. The reaction area 21 is defined on the surface near the distal end of the lower substrate 20 and comprises a hydrophilic layer. The electrode layer 23 is defined on the surface of the lower substrate 20, and comprises a transmitting electrode 231 and a reference electrode 232. The transmitting electrode 231 which is a transmitting end and arranged in accordance with a shape of the lower substrate 20 comprises a distal portion overlapped with the reaction area 21 of the lower substrate 20, and a proximal portion located at the connection area 201 of the lower substrate 20. The reference electrode 232 which is a grounding terminal and arranged separately from the transmitting electrode 231 in accordance with a shape of the lower substrate 20 comprises a distal portion overlapped with the reaction area 21 of the lower substrate 20, and a proximal portion located at the connection area 201 of the lower substrate 20.

The notch 12 recessed from the upper substrate 10 is corresponding to the connection area 201 of the lower substrate 20, and allows the connection area 201 to be positioned on the notch 12. The notch 22 recessed from the lower substrate 20 is corresponding to the connection area 101 of the upper substrate 10, and allows the connection area 101 to be positioned on the notch 22. The connection area 101 of the upper substrate 10 and the connection area 201 of the lower substrate 20 are positioned side by side.

The upper insulation sheet 30 connected to the surface of the upper substrate 10, but excluded from the connection area 101 comprises an upper surface, a lower surface, and a permeation portion 31. The permeation portion 31 is exposed on the upper surface of the upper insulation sheet 30 and on the lower surface of the upper insulation sheet 30 and corresponding to the reaction area 11 of the upper substrate 10. The lower insulation sheet 40 connected to the surface of the lower substrate 20, but excluded from the connection area 201 comprises an upper surface, a lower surface, a permeation portion 41 and a venting air duct 42. The permeation portion 41 is formed on the upper surface of the lower insulation sheet 40 and the lower surface of the lower insulation sheet 40, and corresponding to the reaction area 21 of the lower substrate 20. The venting duct hole 42 is formed across the lower insulation sheet 40 and adjacent to the permeation portion 41 thereof to enhance fluid speed of blood.

The separator 50 connected to the upper insulation sheet 30 and the lower insulation sheet 40 comprises an upper surface, a lower surface, an adhesive layer 51 and a permeation portion 52. The adhesive layer 51 is formed on the upper surface of the separator 50 and on the lower surface thereof, but excluded from the venting air duct 42 and the permeation portion 52. The permeation portion is exposed on the upper surface of the separator 50 and the lower surface of the separator 50 and corresponding to the permeation portion 31 of the upper insulation sheet 30 and permeation portion 41 of the lower insulation sheet 40, respectively.

As performing the invention, connection of the upper substrate 10 and the lower substrate 20 is performed by overlapping the distal portion of the electrode layer 13 with the reaction area 11 of the upper substrate 10 and overlapping the distal portion of the electrode layer 23 with the reaction area 21 of the lower substrate 20, respectively. As dropping the blood on the reaction area 11 or on the reaction area 21, an electron cycle e is formed, and transferring signals to the proximal portion of the connection area 101 of the upper substrate 10 or the connection area 201 of the lower substrate 20. Furthermore, apply an enzyme layer to the reaction area 11 and to the reaction area 21, respectively to examine blood glucose. Connect the upper insulation sheet 30 and the lower insulation sheet 40 to the surface of the upper substrate 10 and the surface of the lower substrate 20, respectively to avoid examining errors caused by contacting the electrode layer 13 of the upper substrate 10 with the electrode layer 23 of the lower substrate 20, so as to make the examining test paper have twice examining function by combining the upper substrate 10 with the lower substrate 20. During the process of blood examination, dropping the blood once on the reaction area 11 of the upper substrate 10 and completing the first examination; furthermore, the blood is permeating at the same time by function of the hydrophilic layer on the reaction area 11, and passing through the permeation portion 31, the permeation portion 41 and the permeation portion 52 to the reaction area 21 of the lower substrate 20. Twice blood glucose examination will be done as the blood distributes completely, and we can confirm the accuracy of blood glucose concentration from the two examining results.

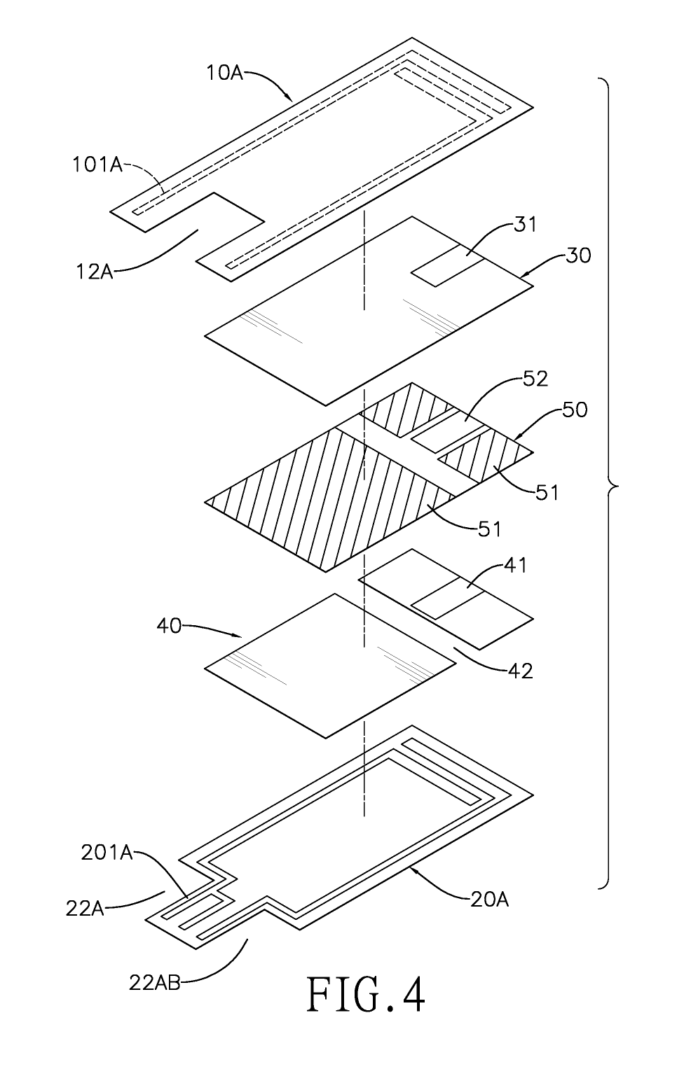

With reference to FIG. 4, the second embodiment in accordance with the present invention is similar to the first embodiment, and comprises an upper substrate 10A, a lower substrate 20A, an upper insulation sheet 30, a lower insulation sheet 40 and a separator 50. The structure of examining test paper used in the second embodiment different from the first embodiment comprises a notch 12, a notch 22A, and an secondary notch 22AB, and a connection area 201A. The notch 12A of the upper substrate 10A is recessed on a central portion of the proximal end of the upper surface of the upper substrate 10A, and the connection area 101A of the upper substrate 10A bilaterally flanks the notch 12A of the upper substrate 10A. The notch 22A of the lower substrate 20A is recessed on a lateral portion of the proximal end of a lower substrate 20A, and the secondary notch 22AB is further recessed on an opposite portion of the proximal end of the lower substrate 20A. The connection area 201A is formed between the notch 22A and the secondary notch 22AB of the lower substrate 20A. The notch 12A of the upper substrate 10A is corresponding to the connection area 201A of the lower substrate 20A, and the connection area 101A of the upper substrate 10A and the connection area 201A of the lower substrate 20A are positioned side by side.

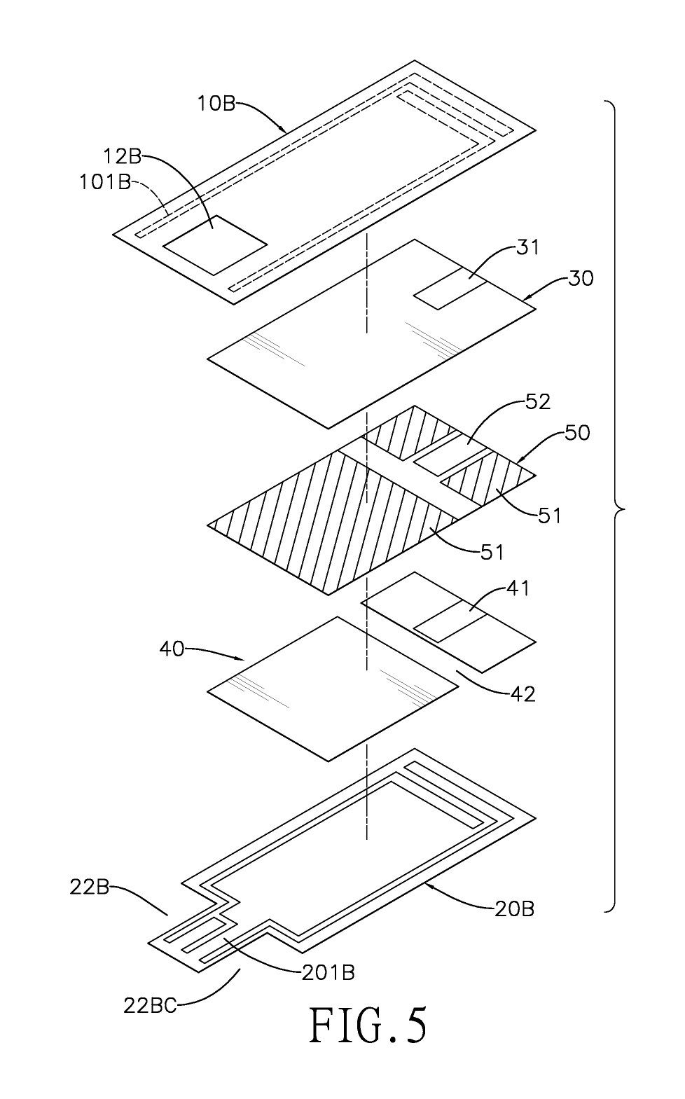

With reference to FIG. 5, the third embodiment in accordance with the present invention is similar to the second embodiment, and comprises an upper substrate 10B, a lower substrate 20B, an upper insulation sheet 30, a lower insulation sheet 40 and a separator 50. The structure of examining test paper used in the third embodiment different from the second embodiment comprises a notch 12B, a notch 22B, a secondary notch 22BC, and a connection are 201B. The notch 12B of the upper substrate 10B is defined through a central region of the upper substrate 10B adjacent to the proximal end, and the connection area 101B of the upper substrate 10B bilaterally flanks the notch 12B of the upper substrate 10B. The notch 22B of the lower substrate 20B is recessed on a lateral portion of the proximal end of a lower substrate 20B, and the secondary notch 22BC is further recessed on an opposite portion of the proximal end of the lower substrate 20B. The connection area 201B is formed between the notch 22B and the secondary notch 22BC of the lower substrate 20B. The notch 12B of the upper substrate 10B is corresponding to the connection area 201B of the lower substrate 20B.

The connection area 101, 101A and 101B formed on the upper substrate 10, 10A and 10B, respectively, in accordance with the present invention are not connected with the connection area 201, 201A and 201B formed on the lower substrate 20, 20A and 20B, but are arranged in alignment separately. During the process of examination, users can separate the two connectors of the two examining test papers at different position for providing connecting points of the blood glucose meter by using a mutual converter.

In a preferable embodiment, a GOD/GDH enzyme layer is applied to the reaction area 11 of the upper substrate 10 and to the reaction area 21 of the lower substrate 20, respectively, and then the reaction area 11 of the upper substrate 10 is set as a capacitive recognition suite, and the reaction area 11 of the upper substrate 10 and the reaction area 21 of the lower substrate 20 are set as an examining electrode suite to enhance the accuracy of blood glucose measurement.

In a preferable embodiment, the GOD/GDH enzyme layer is applied to the reaction area 11 of the upper substrate 10, and an impedance membrane is applied on the reaction area 21 of the lower substrate 20 to enhance the capacitive accuracy of the reaction area 11 and reaction area 21. The reaction 11 of the upper substrate 10 is a DC examining electrode suite, and the reaction 21 of the lower substrate 20 is a AC capacitive recognition suite, and the DC examining electrode suite and the AC capacitive recognition suite are reacting at the same time.

In a more preferable embodiment, the GOD/GDH enzyme layer is applied to the reaction area 11 of the upper substrate 10 to examine blood glucose, and another enzyme is applied to the reaction area 21 of the lower substrate 20 to examine other biochemical substrates (such as cholesterol, blood lactate and uric acid), so that different blood compositions can be examined at the same time in a single examining test paper, and the combination of the enzyme can be chosen by need.

Sentences described above are just preferable examples, but not restricted to the invention; although the preferable examples of the invention had been shown as above, not restricted to the invention; any general knowledge belongs to the technical field, some technical contents described above can be altered or modified slightly to get the same effect of the experimental examples based on specification of the technical program of the invention; however, any technical content described above simple altered or modified to get the same effect of the experimental examples based on specification of the technical program of the invention, belong to the specification of the technical program of the invention.

What is claimed is:

1. An examining test paper comprising:
an upper substrate, being a thin film and comprising:
a surface,
a proximal end,
a distal end opposite to the proximal end,
a notch recessed on the proximal end of the upper substrate,
a connection area formed on the upper substrate adjacent to the notch,
a reaction area defined on the surface near the distal end of the upper substrate, and
an electrode layer defined on the surface of the upper substrate and comprising:
a distal portion overlapped with the reaction area, and
a proximal portion located at the connection area;
a lower substrate, being a thin film and comprising:
a surface,
a proximal end,
a distal end opposite to the proximal end,
a notch recessed on the proximal end of the lower substrate,
a connection area formed on lower substrate adjacent to the notch of the lower substrate,
a reaction area defined on the surface near the distal end of the lower substrate, and
an electrode layer defined on the surface of the lower substrate and comprising:
a distal portion overlapped with the reaction area of the lower substrate, and
a proximal portion located at the connection area of the lower substrate;
an upper insulation sheet connected to the surface of the upper substrate and comprising:
an upper surface,
a lower surface, and
a permeation portion exposed on the upper surface of the upper insulation sheet and the lower surface of the upper insulation sheet and corresponding to the reaction area of the upper substrate;
a lower insulation sheet connected to the surface of the lower substrate and comprising:
an upper surface,
a lower surface, and
a permeation portion exposed on the upper surface of the lower insulation sheet and the lower surface of the lower insulation sheet and corresponding to the reaction area of the lower substrate; and
a separator connected to the upper insulation sheet and the lower insulation sheet and comprising:
an upper surface,
a lower surface, and
a permeation portion exposed on the upper surface of the separator and the lower surface of the separator and respectively corresponding to the permeation portion of the upper insulation sheet and the permeation portion of the lower insulation sheet.

2. The examining test paper as claimed in claim 1, wherein:
the electrode layer of the upper substrate comprises:
a transmitting electrode arranged in accordance with a shape of the upper substrate and comprising:
a distal portion overlapped with the reaction area of the upper substrate, and
a reference electrode arranged separately from the transmitting electrode in accordance with a shape of the upper substrate and comprising:
a distal portion overlapped with the reaction area of the upper substrate, and the electrode layer of the lower substrate comprises:
a transmitting electrode arranged in accordance with a shape of the lower substrate and comprising:
a distal portion overlapped with the reaction area of the lower substrate, and
a reference electrode arranged separately from the transmitting electrode of the lower substrate in accordance with a shape of the lower substrate and comprising:
a distal portion overlapped with the reaction area of the lower substrate.

3. The examining test paper as claimed in claim 1, wherein the notch of the upper substrate is corresponding to the connection area of the lower substrate; and
the notch of the lower substrate is corresponding to the connection area of the upper substrate.

4. The examining test paper as claimed in claim 2, wherein the notch of the upper substrate is corresponding to the connection area of the lower substrate; and
the notch of the lower substrate is corresponding to the connection area of the upper substrate.

5. The examining test paper as claimed in claim 1, wherein the reaction area of the upper substrate comprises a hydrophilic layer; and
the reaction area of the lower substrate comprises a hydrophilic layer.

6. The examining test paper as claimed in claim 5, wherein the separator comprises:
an upper adhesive layer defined on the upper surface of the separator and attached to a lower surface of the upper insulation sheet, and
an lower adhesive layer defined on the lower surface of the separator and attached to an upper surface of the lower insulation sheet.

7. The examining test paper as claimed in claim 6, wherein the lower insulation sheet comprises a venting duct formed across the lower insulation sheet and adjacent to the permeation portion thereof.

8. The examining test paper as claimed in claim 7, wherein the notch of the upper substrate is recessed on a central portion of the proximal end of the upper surface of the upper substrate, and the connection area of the upper substrate bilaterally flanks the notch of the upper substrate;
the notch of the lower substrate is recessed on a lateral portion of the proximal end of the lower substrate;
the lower substrate further comprises a secondary notch recessed on an opposite portion of the proximal end of the lower substrate; and
the connection area of the lower substrate is formed between the notch and the secondary notch of the lower substrate, wherein the notch of the upper substrate is corresponding to the connection area of the lower substrate.

9. The examining test paper as claimed in claim 7, wherein the notch of the upper substrate is defined through a central region of the upper substrate adjacent to the proximal end, and the connection area of the upper substrate bilaterally flanks the notch of the upper substrate;
the notch of the lower substrate is recessed on a bilateral part portion of the proximal end of the lower substrate;
the lower substrate further comprises a secondary notch recessed on an opposite part of the proximal end of the lower substrate; and
the connection area of the lower substrate is formed between the notch and the secondary notch of the lower substrate, wherein the notch of the upper substrate is corresponding to the connection area of the lower substrate.
